(19) United States
(12) Patent Application Publication
(10) Pub. No.: US 2026/0347751 A1
(43) Pub. Date: Nov. 5, 2026

(54) Yeast Strains Having High Protein Content

(71) Applicant: MICROBIOGEN PTY LTD, Sydney, New South Wales (AU)

(72) Inventors: Philip John Livingstone BELL, Turramurra, New South Wales (AU); Sophia MANDARAKAS, West Ryde, New South Wales (AU)

(73) Assignee: MICROBIOGEN PTY LTD, Sydney, New South Wales (AU)

(21) Appl. No.: 19/485,183

(22) PCT Filed: May 17, 2024
(86) PCT No.: PCT/AU2024/050496

§ 371 Date: Nov. 17, 2025

(30) Foreign Application Priority Data
May 19, 2023 (AU) 2023901560

(51) Int. Cl. C12N 1/185; A23J 1/18; A23K 10/18; A23K 20/147; C12R 1/865

(52) U.S. Cl. CPC C12N 1/185; A23J 1/18; A23K 10/18; A23K 20/147; C12R 2001/865

(57) ABSTRACT

The present invention relates to yeast strains and their use. In particular, the present invention relates to strains of yeast strains that express high levels of protein when growing on industrial waste, and the use of such strains in animal feed preparations.

### TECHNICAL FIELD

The present application claims priority from Australian Provisional Patent Application No. 2023901560 (filed 19 May 2023), the contents of which are incorporated in their entirety herein.

The present invention relates to yeast strains and their use. In particular, the present invention relates to strains of yeast strains that express high levels of protein when growing on industrial waste, and the use of such strains in animal feed preparations.

### BACKGROUND

Any discussion of the prior art throughout the specification should in no way be considered as an admission that such prior art is widely known or forms part of common general knowledge in the field.

Yeast of the species Saccharomyces are widely used in animal feed applications where they can be either probiotic or simply a readily digestible source of protein and nucleic acids.

Yeast which are used for production of animal feed, require several characteristics to ensure cost effective production of high protein animal feed. These characteristics include high growth rates, high yield, efficient nitrogen utilisation, and the ability to achieve high levels of protein when grown in fed-batch culture. In these fed-batch processes, the substrate costs represent a very significant component of the costs of manufacture. Thus, the listed characteristics have a marked effect of the viability of the industrial process.

Currently strains of Saccharomyces cerevisiae used in the production of animal feed are limited in the sorts of carbon that can be economically used as a substrate for growth. The majority of Saccharomyces biomass produced worldwide is grown in Fed-batch culture using either molasses where the sugar source is sucrose, or corn syrups where the sugar source is glucose. Although molasses is a by-product from the sugar manufacturing industry, it has significant value as an animal feed, and also as a substrate to many industrial processes that require the production of microbial biomass. As a result, molasses can be in demand and subject to increases in price, which affects the economics of producing Saccharomyces as a protein source. Similarly, corn syrup is used in many human food applications.

Although strains currently used in the animal feed industry are well suited to biomass production, there is an increasing need to obtain yeasts of the genus Saccharomyces that efficiently use carbon sources other than sucrose or glucose, yet be able to produce yeast with high protein content suitable for animal feed. For example, waste high in glycerol is produced as by-products from both bio-diesel production and corn ethanol production. In addition, paper pulp manufacture using the sulfite process produces large quantities of waste rich in xylose and in acetic acid.

In the case of glycerol as a substrate for yeast biomass production, it is widely reported that yeast of genus Saccharomyces can grow slowly on glycerol, provided addition nutrients, such yeast extract, are provided. The addition of yeast extract to an industrial process to produce yeast biomass is prohibitively expensive, and thus it would be advantageous to develop strains of Saccharomyces that can utilise glycerol at high yield and productivity without the addition of expensive nutrients such as yeast extract.

In the case of xylose as a substrate for yeast biomass production, it is widely reported that the yeast of the genus Saccharomyces are extremely poor at utilising xylose, and thus for many years it was considered that Saccharomyces could not utilise xylose at all.

It would therefore be advantageous to develop new yeast strains capable of growing efficiently on a wide range of alternative carbon sources such as xylose, glycerol, acetate, lactic acid, and ethanol whilst simultaneously producing high levels of protein in conditions that are sub-optimal for both growth and protein production.

It is therefore an object of the present invention to overcome or ameliorate at least one of the disadvantages of the prior art, or to provide a useful alternative.

### SUMMARY

The inventors have developed a non-genetically engineered high protein strain of Saccharomyces which grows efficiently utilising industrial waste substrates that comprise alternative carbon sources such as glycerol, xylose, acetate, lactic acid and ethanol, and is suitable for use in animal feed.

A first aspect of the present invention provides a Saccharomyces strain comprising the following characteristics:
(i) capable of growing on industrial waste with efficiency about the same as the Saccharomyces strain deposited under the Budapest Treaty and having NMI accession no. V23/009171 (strain MBG 4917) when grown under the same conditions; and
(ii) containing about the same amount of protein as strain MBG 4917 when grown under the same conditions

A second aspect of the present invention provides a Saccharomyces strain selected from:
(a) the Saccharomyces strain deposited under the Budapest Treaty and having NMI accession no. V23/009171 (strain MBG 4917); and
(b) a derivative of strain MBG 4917.

A third aspect of the present invention provides a Saccharomyces strain capable of growing on industrial waste and having a protein content of at least 50% by weight.

In one embodiment, the industrial waste is selected from the group consisting of corn ethanol stillage, palm kernel hydrolysate and biodiesel derived glycerol.

A fourth aspect of the present invention provides a Saccharomyces strain capable of growing on xylose, glycerol, acetate, lactic acid and/or ethanol, and having a protein content of at least 50% by weight.

In one embodiment, the protein content of the Saccharomyces strain is in the range of about 50% to about 70% by weight.

In one embodiment the protein content of the Saccharomyces strain is at least about 50%, is at least about 51%, is at least about 52%, is at least about 53%, is at least about 54%, is at least about 55%, is at least about 56%, is at least about 57%, is at least about 58%, is at least about 59%, is at least about 60%, is at least about 61%, is at least about 62%, is at least about 63%, is at least about 64%, is at least about 65%, is at least about 66%, is at least about 67%, or is at least about 68%, is at least about 69%.

In one embodiment the protein content of the Saccharomyces strain is about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, or about 69%.

In one embodiment, the Saccharomyces strain is a non-genetically engineered Saccharomyces strain.

In one embodiment, the Saccharomyces strain is the strain deposited under the Budapest Treaty and having NMI accession no. V23/009171 (strain MBG 4917).

A fifth aspect of the present invention provides the Saccharomyces strain deposited under the Budapest Treaty and having NMI accession no. V23/009171 (strain MBG 4917).

A sixth aspect of the present invention provides a method of producing yeast having a protein content of at least 50% by weight, comprising incubating the Saccharomyces strain according to any one of the first to fifth aspects with a substrate comprised of industrial waste, under conditions which promote growth of the Saccharomyces strain.

In one embodiment, the industrial waste is selected from the group consisting of corn ethanol stillage, palm kernel hydrolysate and biodiesel derived glycerol.

A seventh aspect of the present invention provides a method of producing yeast having a protein content of at least 50% by weight, comprising incubating the Saccharomyces strain according to any one of the first to fifth aspects with a substrate comprising xylose, glycerol, acetate, lactic acid and/or ethanol, under conditions which promote growth of the Saccharomyces strain.

In one embodiment, the protein content of the yeast is in the range of about 50% to about 70% by weight.

In one embodiment the protein content of the yeast is at least about 50%, is at least about 51%, is at least about 52%, is at least about 53%, is at least about 54%, is at least about 55%, is at least about 56%, is at least about 57%, is at least about 58%, is at least about 59%, is at least about 60%, is at least about 61%, is at least about 62%, is at least about 63%, is at least about 64%, is at least about 65%, is at least about 66%, is at least about 67%, or is at least about 68%, is at least about 69%.

In one embodiment the protein content of the yeast is about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, or about 69%.

In one embodiment, the Saccharomyces strain is capable of growing on xylose.

In one embodiment, the Saccharomyces strain is capable of growing on xylose as the sole carbon source.

In one embodiment, the Saccharomyces strain grows on xylose at a rate that is at least 100 times faster than Ethanol Red at 48 hours.

In one embodiment, the Saccharomyces strain grows on xylose at a rate that is at least 100 times faster than Ethanol Red at 48 hours under the conditions of Test T1.

In one embodiment, the Saccharomyces strain grows on xylose at a rate that is about 130 times faster than Ethanol Red at 48 hours under the conditions of Test T1.

In one embodiment, the Saccharomyces strain is capable of growing on glycerol.

In one embodiment, the Saccharomyces strain is capable of growing on glycerol as the sole carbon source.

In one embodiment, the Saccharomyces strain grows on glycerol at a rate that is at least 50 times faster than Ethanol Red at 48 hours.

In one embodiment, the Saccharomyces strain grows on glycerol at a rate that is at least 50 times faster than Ethanol Red at 48 hours under the conditions of Test T2.

In one embodiment, the Saccharomyces strain grows on glycerol at a rate that is about 58 times faster than Ethanol Red at 48 hours under the conditions of Test T2.

In one embodiment, the Saccharomyces strain is capable of growing on acetate.

In one embodiment, the Saccharomyces strain is capable of growing on acetate as the sole carbon source.

In one embodiment, the Saccharomyces strain grows on acetate at a rate that is at least 15 times faster than Ethanol Red at 48 hours.

In one embodiment, the Saccharomyces strain grows on acetate at a rate that is at least 15 times faster than Ethanol Red at 48 hours under the conditions of Test T3.

In one embodiment, the Saccharomyces strain grows on acetate at a rate that is about 19 times faster than Ethanol Red at 48 hours under the conditions of Test T3.

In one embodiment, the Saccharomyces strain is capable of growing on lactic acid.

In one embodiment, the Saccharomyces strain is capable of growing on lactic acid as the sole carbon source.

In one embodiment, the Saccharomyces strain grows on lactic acid at a rate that is at least 5 times faster than Ethanol Red at 48 hours.

In one embodiment, the Saccharomyces strain grows on lactic acid at a rate that is at least 5 times faster than Ethanol Red at 48 hours under the conditions of Test T4.

In one embodiment, the Saccharomyces strain grows on lactic acid at a rate that is about 7 times faster than Ethanol Red at 48 hours under the conditions of Test T4.

In one embodiment, the Saccharomyces strain is capable of growing on ethanol.

In one embodiment, the Saccharomyces strain is capable of growing on ethanol as the sole carbon source.

In one embodiment, the Saccharomyces strain grows on ethanol at a rate that is at least 5 times faster than Ethanol Red at 48 hours.

In one embodiment, the Saccharomyces strain grows on ethanol at a rate that is at least 5 times faster than Ethanol Red at 48 hours under the conditions of Test T5.

In one embodiment, the Saccharomyces strain grows on ethanol at a rate that is about 7 times faster than Ethanol Red at 48 hours under the conditions of Test T5.

In one embodiment, the Saccharomyces strain is capable of growing on corn ethanol stillage.

An eighth aspect of the present invention provides a yeast having a protein content of at least 50% by weight produced by the method according to the sixth aspect or seventh aspect.

In one embodiment, the protein content of the yeast is in the range of about 50% to about 70% by weight.

In one embodiment the protein content of the yeast is at least about 50%, is at least about 51%, is at least about 52%, is at least about 53%, is at least about 54%, is at least about 55%, is at least about 56%, is at least about 57%, is at least about 58%, is at least about 59%, is at least about 60%, is at least about 61%, is at least about 62%, is at least about 63%, is at least about 64%, is at least about 65%, is at least about 66%, is at least about 67%, or is at least about 68%, is at least about 69%.

In one embodiment the protein content of the yeast is about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, or about 69%.

A ninth aspect of the present invention provides an animal feed composition comprising the Saccharomyces strain according to any one of the first to fifth aspects or the yeast according to the eighth aspect.

A tenth aspect of the present invention provides a method of producing yeast having a protein content of at least 50% by weight, comprising incubating the Saccharomyces strain according to any one of the first to fifth aspects with corn ethanol stillage, under conditions which promote growth of the Saccharomyces strain.

In one embodiment, the protein content of the yeast is in the range of about 50% to about 70% by weight.

In one embodiment the protein content of the yeast is at least about 50%, is at least about 51%, is at least about 52%, is at least about 53%, is at least about 54%, is at least about 55%, is at least about 56%, is at least about 57%, is at least about 58%, is at least about 59%, is at least about 60%, is at least about 61%, is at least about 62%, is at least about 63%, is at least about 64%, is at least about 65%, is at least about 66%, is at least about 67%, or is at least about 68%, is at least about 69%.

In one embodiment the protein content of the yeast is about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, or about 69%.

In one embodiment, the Saccharomyces strain grown on corn ethanol stillage produces about 0.4 g per g of available substrate.

In one embodiment, the Saccharomyces strain grows on corn ethanol stillage at a rate of about 2.3 g yeast produced per Litre per hour.

In one embodiment, the protein content of the Saccharomyces strain grown on corn ethanol stillage is about 58%.

In one embodiment, the total utilisable carbon for the Saccharomyces strain grown on corn ethanol stillage is about 10% w/v to about 20% w/v.

In one embodiment, the protein yield for the Saccharomyces strain grown on corn ethanol stillage is about 0.3 g yeast per g of total utilizable carbon to about 0.4 g yeast per g of total utilizable carbon.

In one embodiment, the protein content for the Saccharomyces strain grown on corn ethanol stillage is about 58% to about 60%.

In one embodiment, the Saccharomyces strain is capable of growing on biodiesel derived glycerol.

An eleventh aspect of the present invention provides a method of producing yeast having a protein content of at least 50% by weight, comprising incubating the Saccharomyces strain according to any one of the first to fifth aspects with biodiesel derived glycerol, under conditions which promote growth of the Saccharomyces strain.

In one embodiment, the protein content of the yeast is in the range of about 50% to about 70% by weight.

In one embodiment the protein content of the yeast is at least about 50%, is at least about 51%, is at least about 52%, is at least about 53%, is at least about 54%, is at least about 55%, is at least about 56%, is at least about 57%, is at least about 58%, is at least about 59%, is at least about 60%, is at least about 61%, is at least about 62%, is at least about 63%, is at least about 64%, is at least about 65%, is at least about 66%, is at least about 67%, or is at least about 68%, is at least about 69%.

In one embodiment the protein content of the yeast is about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, or about 69%.

In one embodiment, the Saccharomyces strain grown on biodiesel derived glycerol produces about 0.4 g per g of available substrate.

In one embodiment, the Saccharomyces strain grows on biodiesel derived glycerol at a rate of about 3.1 g yeast produced per Litre per hour.

In one embodiment, the protein level in the Saccharomyces strain grown on biodiesel derived glycerol is about 60%.

In one embodiment, the total utilisable carbon for the Saccharomyces strain grown on biodiesel derived glycerol/cane molasses blend is about 30% w/v.

In one embodiment, the protein yield for the Saccharomyces strain grown on biodiesel derived glycerol/cane molasses blend is about 0.4 g yeast per g of total utilizable carbon to about 0.5 g yeast per g of total utilizable carbon.

In one embodiment, the protein content for the Saccharomyces strain grown on biodiesel derived glycerol/cane molasses blend is about 53% to about 65%.

A twelfth aspect of the present invention provides a method of producing yeast having a protein content of at least 50% by weight, comprising incubating the Saccharomyces strain according to any one of the first to fifth aspects with palm kernel hydrolysate, under conditions which promote growth of the Saccharomyces strain.

In one embodiment, the protein content of the yeast is in the range of about 50% to about 70% by weight.

In one embodiment the protein content of the yeast is at least about 50%, is at least about 51%, is at least about 52%, is at least about 53%, is at least about 54%, is at least about 55%, is at least about 56%, is at least about 57%, is at least about 58%, is at least about 59%, is at least about 60%, is at least about 61%, is at least about 62%, is at least about 63%, is at least about 64%, is at least about 65%, is at least about 66%, is at least about 67%, or is at least about 68%, is at least about 69%.

In one embodiment the protein content of the yeast is about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, or about 69%.

In one embodiment, the Saccharomyces strain grown on palm kernel hydrolysate produces about 0.45 g yeast per g of the available substrate.

In one embodiment, the Saccharomyces strain grows on palm kernel hydrolysate at a rate of about 3.7 g yeast produced per Litre per hour.

In one embodiment, the protein level in the Saccharomyces strain grown on palm kernel hydrolysate is about 58%.

In one embodiment, the total utilisable carbon for the Saccharomyces strain grown on palm kernel hydrolysate is about 10-15% w/v.

In one embodiment, the protein yield for the Saccharomyces strain grown on palm kernel hydrolysate is about 0.3 g yeast per g of total utilizable carbon to about 0.5 g yeast per g of total utilizable carbon.

In one embodiment, the protein content for the Saccharomyces strain grown on palm kernel hydrolysate is about 58% to about 65%.

In one embodiment, a derivative of strain MBG 4917 is a hybrid strain produced by culturing a first yeast strain with strain MBG 4917 under conditions which permit combining of DNA between the first yeast strain and strain MBG 4917.

In one embodiment, the derivative of strain MBG 4917 exhibits all the characteristics of strain MBG 4917.

In one embodiment, the derivative of strain MBG 4917 may be prepared by culturing a first yeast strain with strain MBG 4917, under conditions which permit combining of DNA between the first yeast strain and strain MBG 4917.

In one embodiment, culturing the first yeast strain with strain MBG 4917, under conditions which permit combining of DNA between the first yeast strain and strain MBG 4917, comprises:
(i) sporulating the first yeast strain and strain MBG 4917;
(ii) germinating and hybridizing spores produced by the first yeast strain with spores produced by strain MBG 4917.

Methods for sporulating, germinating and hybridising yeast strains, and in particular, Saccharomyces strains, are known in the art and are described in, for example, Ausubel et al. 1997, Current Protocols in Molecular Biology, Volume 2, pages 13.2.1 to 13.2.5 (John Willey & Sons Inc) and Chapter 7, “Sporulation and Hybridisation of yeast” by R. R. Fowell, in “The Yeasts” vol 1, A. H. Rose and J. S. Harrison (Eds), 1969, Academic Press.

In one embodiment, the yeast strains may be cultured under conditions which permit cell fusion. Methods for the generation of intraspecific or interspecific hybrids using cell fusion techniques are described in, for example, Spencer et al. (1990) in, Yeast Technology, Spencer JFT and Spencer DM (Eds), Springer Verlag, New York.

In another embodiment, the yeast strains may be cultured under conditions which permit cytoduction. Methods for cytoduction are described in, for example, Inge-Vechymov et al. (1986) Genetika 22:2625-2636; Johnston (1990) in, Yeast technology, Spencer JFT and Spencer DM (Eds), Springer Verlag, New York.

In one embodiment, a derivative of strain MBG 4917 may be a mutant of this strain. Methods for producing mutants of Saccharomyces yeast, and specifically mutants of Saccharomyces cerevisiae, are known in the art and described in, for example, Lawrence C. W. (1991) Methods in Enzymology, 194:273-281.

### DEFINITIONS

In describing and claiming the present invention, the following terminology has been used in accordance with the definitions set out below. It is also to be understood that the terminology used herein is for the purpose of describing embodiments of the invention only and is not intended to be limiting. Unless defined otherwise, all technical and scientific terms used herein have the same meaning as commonly understood by one having ordinary skill in the art to which the invention pertains.

As used herein the term “about” can mean within 1 or more standard deviation per the practice in the art. Alternatively, “about” can mean a range of up to 10%. When values are provided in the specification and claims the meaning of “about” should be assumed to be within an acceptable error range for that value.

In the context of the present invention, the words “comprise”, “comprising” and the like are to be construed in their inclusive, as opposed to their exclusive, sense, that is in the sense of “including, but not limited to”.

The terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

Other than in the operating examples, or where otherwise indicated, all numbers expressing quantities of ingredients or reaction conditions used herein are to be understood as modified in all instances by the term ‘about’.

The recitation of a numerical range using endpoints includes all numbers subsumed within that range (e.g., 1 to 5 includes 1, 1.5, 2, 2.75, 3, 3.80, 4, 5, etc.).

As used herein, the term “industrial waste” means fluid and/or aqueous residues and by-products from industrial processes such as corn ethanol plants, biodiesel plants, paper pulp plants and similar processes. In the context of the present invention the phrases “industrial waste” and “industrial waste streams” may be used interchangeably.

As used herein, the term “efficiency” in the context of yeast growth means growth of yeast at a rate comparable to growth rate in medium that promotes optimal or near optimal growth of yeast.

As used herein, the terms “high protein content” or “high protein levels” means 50% or above protein on a dry matter basis.

As used herein, the term “derivative” means a yeast that has the same or similar phenotype as the yeast of the present invention that is derived by breading from and/or mutation of the yeast of the invention or is a genetically engineered version of the yeast of the present invention, having the same or similar phenotype.

As used herein, the term “alternative carbon source” means a source of carbon that is not a sugar.

As used herein, the term “non-genetically engineered” refers to strains of yeast that have not undergone genetic modification using techniques such as gene insertion, gene knockout, gene editing, or transgenic technology. Instead, these yeast strains have been developed or bred using traditional methods of selection, hybridization, or mutation breeding to achieve desired characteristics or traits.

### BRIEF DESCRIPTION OF THE INVENTION

The inventors recognized that it would be advantageous to grow yeast on readily available, low-cost substrates such as by-products of large-scale industrial processes, to produce yeast having high protein content suitable for animal feed.

Thus, the present invention is concerned with a non-genetically engineered Saccharomyces strain having high protein content suitable for addition to animal feed, which can grow on substrates currently considered not to be suitable for, or optimal for, production of yeast at industrial levels and having a suitably high protein content for application in animal feed.

The substrates that the yeast of the present invention can utilise effectively comprise industrial waste such as those from biodiesel/corn ethanol production, and paper pulp production, which are rich in glycerol, xylose and/or acetic acid.

Currently industrial processes for production of yeast having high protein content require optimal conditions, which necessitate use of carbon sources such as molasses or corn syrup can provide. The yeast of the present invention can be grown on waste substrates and still produce protein levels equal to, or superior to, conventionally grown yeast using molasses or sugar syrups.

The strain MBG 4917 was produced via an evolution and breeding program that selected for yeast capable of growing on a variety of carbon sources including xylose, glycerol and/or acetate as sole carbon source. The strain has been developed entirely using methods that do not involve genetic engineering.

Strain MBG 4917 has been deposited under Budapest Treaty at the National Measurement Institute (NMI), 1/153 Bertie Street, Port Melbourne, Victoria, Australia 3207 on 17 May 2023 and having NMI accession number V23/009171.

The yeast of the present invention having high protein content is suitable as an additive to a variety of animal feed formulations which require enhanced protein content sources having easy digestibility properties and low production cost. Thus, the yeast of then present invention can be added to animal feed formulations designed for fish, poultry, swine and other domestic and farm animals. Suitable animal feed formulations are covered extensively in scientific and technical literature, and would be known to those skilled in the art (see, e.g., Animal Feed Formulations, Pesti & Miller, Springer Nature B.V., 1993; Basic Animal Nutrition and Feeding, Pond et al., John Wiley & Sons Inc., 2004; Developing Animal Feed Products, Partheeban (Ed.), Burleigh Dodds Science Publishing Limited, 2021; Agboola et al. 2021, Reviews in Aquaculture 13:949-970; Abass et al. 2018, Aquaculture International 26:843-855; Ozório et al. 2012, Animals 2:16-24; Aghdamshahriar et al. “The effect of yeast (Saccharomyces cerevisiae) in replacement fish meal and poultry by product protein in broiler diets.” XII European Poultry Conference, Verona, Italia. 2006; Ly et al. 2017, Journal of Agricultural Science and Technology A 7:345-49; Winkler et al. 2011, Livestock Science 137:168-177; Maamouri & Ben Salem 2022, Veterinary Medicine and Science 8:398-404).

The practice of the present invention employs, unless otherwise indicated, conventional microbiology and classical genetics. Such techniques are known to the skilled worker and are explained fully in the literature. See, for example, Sherman et al. “Methods in Yeast Genetics” (1981) Cold Spring Harbor Laboratory Manual, Cold Spring Harbor, New York; European Patent number EP 0 511 108 B.

To exemplify the nature of the present invention such that it may be more clearly understood, the following non-limiting examples are provided.

### EXAMPLES

### Example 1: Growth of Strain MBG 4917 on Xylose as a Sole Carbon Source (Test T1)

Strain MBG 4917 is the product of a breeding program to generate Saccharomyces cerevisiae strains that grow efficiently utilising industrial waste substrates that comprise alternative carbon sources such as glycerol, xylose, acetate, lactic acid and ethanol. Such a phenotype is not found in naturally occurring Saccharomyces strains.

Strain MBG 4917 was inoculated into 50 mL of broth consisting of 5% xylose and 1× Yeast Nitrogen Base (YNB, Difco) in a 250 mL Ehrlenmeyer flask at an Optical density (OD) of ~0.1 which is approximately 1×10⁶ cells per mL. The YNB contains inorganic yeast nutrients and small quantities of essential vitamins. To maintain the pH within an acceptable range for growth of Saccharomyces, the minimal medium broth was buffered with 0.3 g/100 mL citric acid and 0.7 g/100 mL trisodium citrate, and the pH is adjusted to pH 5 using either NaOH or HCl (strain MBG 4917 is incapable of using citrate as a carbon source). The cultures were incubated at 30 °C in an orbital shaker rotating at 200 rpm to ensure suspension of the cells and maintain aeration. At 24 and 48 hr the OD of the cultures was measured so that any growth can be quantified.

The growth of strain MBG 4917 was compared with the growth of a wildtype industrial strain of Saccharomyces cerevisiae, Ethanol Red (ER, obtained from LeSaffre Yeast Corporation, USA).

TABLE 1

| | OD (5% Xylose) | | |
|---|---|---|---|
| | 0 hr | 24 hr | 48 hr |
| MBG 4917 | 0.135 | 4.35 | 26.4 |
| Ethanol Red | 0.122 | 0.12 | 0.2 |

The results demonstrate that strain MBG 4917 can grow rapidly on xylose as the sole carbon source while Ethanol Red cannot.

### Example 2: Growth MBG 4917 on Glycerol as a Sole Carbon Source (Test T2)

Strain MBG 4917 was inoculated into 50 mL of broth consisting of 3% glycerol and 1×YNB in a 250 mL Ehrlenmeyer flask at an OD of ~0.1 which is approximately 1×10⁶ cells per mL. To maintain the pH within an acceptable range for growth of Saccharomyces, the minimal medium broth was buffered with 0.3 g/100 mL citric acid and 0.7 g/100 mL trisodium citrate, and the pH is adjusted to pH 5 using either NaOH or HCl. The cultures were incubated at 30 °C in an orbital shaker rotating at 200 rpm to ensure suspension of the cells and maintain aeration. At 24 and 48 hr the OD of the cultures was measured so that any growth can be quantified.

The growth of strain MBG 4917 was compared with the growth of a wildtype industrial strain of Saccharomyces cerevisiae (Ethanol Red).

TABLE 2

| | OD (3% glycerol) | | |
|---|---|---|---|
| | 0 hr | 24 hr | 48 hr |
| MBG 4917 | 0.139 | 8.00 | 18.8 |
| Ethanol Red | 0.159 | 0.19 | 0.32 |

The results demonstrate that strain MBG 4917 can grow rapidly on glycerol as the sole carbon source while Ethanol Red cannot.

### Example 3: Growth of MBG 4917 on Acetate as a Sole Carbon Source (Test T3)

Strain MBG 4917 was inoculated into 50 mL of broth consisting of 0.5% acetate and 1×YNB in a 250 mL Erlenmeyer flask at an OD of ~0.1 which is approximately 1×10⁶ cells per mL. To maintain the pH within an acceptable range for growth of Saccharomyces, the minimal medium broth was buffered with 0.3 g/100 mL citric acid and 0.7 g/100 mL trisodium citrate, and the pH is adjusted to pH 5 using either NaOH or HCl. The cultures were incubated at 30 °C in an orbital shaker rotating at 200 rpm to ensure suspension of the cells and maintain aeration. At 24 and 48 hr the OD of the cultures was measured so that any growth can be quantified.

The growth of strain MBG 4917 was compared with the growth of a wild type industrial strain of Saccharomyces cerevisiae (Ethanol Red).

TABLE 3

| | OD (0.5% acetate) | | |
|---|---|---|---|
| | 0 hr | 24 hr | 48 hr |
| MBG 4917 | 0.154 | 6.90 | 5.8 |
| Ethanol Red | 0.138 | 0.17 | 0.3 |

The results demonstrate that strain MBG 4917 can grow on acetate as the sole carbon source while Ethanol Red cannot.

### Example 4: Growth of MBG 4917 on Lactic Acid as a Sole Carbon Source (Test T4)

Strain MBG 4917 was inoculated into 50 mL of broth consisting of 3% lactic acid and 1×YNB in a 250 mL Erlenmeyer flask at an OD of ~0.1 which is approximately 1×10⁶ cells per mL. To maintain the pH within an acceptable range for growth of Saccharomyces, the minimal medium broth was buffered with 0.3 g/100 mL citric acid and 0.7 g/100 mL trisodium citrate, and the pH is adjusted to pH 5 using either NaOH or HCl. The cultures were incubated at 30 °C in an orbital shaker rotating at 200 rpm to ensure suspension of the cells and maintain aeration. At 24 and 48 hr the OD of the cultures was measured so that any growth can be quantified.

The growth of strain MBG 4917 was compared with the growth of a wildtype industrial strain of Saccharomyces cerevisiae (Ethanol Red).

TABLE 4

| | OD (3% lactic acid) | | |
|---|---|---|---|
| | 0 hr | 24 hr | 48 hr |
| MBG 4917 | 0.108 | 0.36 | 1.4 |
| Ethanol Red | 0.142 | 0.20 | 0.2 |

The results demonstrate that strain MBG 4917 can grow on lactic acid as the sole carbon source while Ethanol Red cannot.

### Example 5: Growth of MBG 4917 on Ethanol as a Sole Carbon Source (Test T5)

Strain MBG 4917 was inoculated into 50 mL of broth consisting of 0.5% ethanol in a 250 mL Erlenmeyer flask at an OD ~0.1 which is approximately 1×10⁶ cells per mL To maintain the pH within an acceptable range for growth of Saccharomyces, the minimal medium broth was buffered with 0.3 g/100 mL citric acid and 0.7 g/100 mL trisodium citrate, and the pH is adjusted to pH 5 using either NaOH or HCl. The cultures were incubated at 30 °C in an orbital shaker rotating at 200 rpm to ensure suspension of the cells and maintain aeration. At 24 and 48 hr the OD of the cultures was measured so that any growth can be quantified.

The growth of strain MBG 4917 was compared with the growth of a wildtype industrial strain of Saccharomyces cerevisiae (Ethanol Red).

TABLE 5

| | OD (0.5% ethanol) | | |
|---|---|---|---|
| | 0 hr | 24 hr | 48 hr |
| MBG 4917 | 0.144 | 9.20 | 14.8 |
| Ethanol red | 0.152 | 0.49 | 1.9 |

The results demonstrate that strain MBG 4917 can grow rapidly on ethanol as the sole carbon source while Ethanol Red cannot.

### Example 6: Yeast Growth and Protein Production

Examples 1 to 5 show that MBG 4917 can grow on xylose, glycerol, acetate, lactic acid and/or ethanol as the sole carbon source, demonstrating that the strain can grow on industrial waste.

To demonstrate the ability of strain MBG 4917 to reach high protein levels when grown on industrial waste, experiments were conducted using a fed-batch process where feeding is done in a continuous manner to sustain the growth of yeast by which higher cell densities can be achieved compared to a batch process. The process started with the addition of nutrients (nitrogen in the form of urea and phosphate in the form of monoammonium phosphate plus minerals and trace metals) and a small amount of substrate (feed-yeast and utilisable carbon compounds) in the set water prior to inoculation of the yeast. Once inoculated, the following parameters were maintained in the fermenter:
Starting volume: 350 mL
Temperature: 30 °C
pH: 4.0-6.0
Air flow rate: 2 litre per minute.
Feed volume: 400 mL

The feed was started around 2-3 hours from the start of the process (inoculation). The feed rate was then controlled in such a way that it is continuous and increasing so that it can sustain the growth of the increasing yeast biomass. When feed was finished, the yeast was allowed to utilise any remaining carbon compound in the fermenter after which the process is ended. The whole process usually took around 20 to 30 hours to finish.

### Growth on Corn Ethanol Stillage

Corn ethanol plants use yeast to ferment the starch from corn into ethanol. During this process, the yeast produces glycerol as a by-product. Although the glycerol produced is about 10 times less than amount of ethanol produced during a typical corn ethanol fermentation, the large industrial scale of the US corn ethanol industry ensures that considerable quantities of glycerol by-product is produced by the industry. After the removal of the ethanol via distillation, the remaining ‘stillage’ is concentrated in multiple effect evaporators generating a waste syrup rich in glycerol:

TABLE 6

| Substrate | % w/v |
|---|---|
| Glucose | 0.76 |
| Glycerol | 14.11 |
| Fructose | 0.89 |
| Maltose | 3.19 |
| Lactic acid | 0.95 |
| Acetic acid | 0.11 |

MBG 4917 was grown on the waste syrup from a corn ethanol in a fed batch process. At the end of the fed-batch process the yeast was harvested and analysed. MBG 4917 produced 0.38 g of yeast per g of available substrate, grew at a rate of 2.37 g yeast produced per Litre per hour, and the protein level was measured at 58.2%.

### Growth on Crude Biodiesel-Derived Glycerol

Biodiesel is a renewable fuel derived by trans-esterifying plant or animal oils with an alcohol such as either ethanol or methanol. Due to the large quantities of biodiesel produced, and the expense of purifying the glycerol into a form suitable for other applications, the biodiesel derived glycerol can be considered an unwanted by-product. The high biological oxygen demand (BOD) of the glycerol waste stream makes it expensive to dispose of and thus it would be useful to upgrade the waste stream into higher value Saccharomyces biomass.

Strain MBG 4917 was grown on vegetable and animal derived waste glycerol from a biodiesel plant diluted to a concentration of 30% w/v and supplemented with standard additions of trace elements, vitamins, and inorganic nutrients such as Mono-ammonium phosphate (MAP) and urea.

At the end of the fed-batch process the yeast was harvested and analysed.

MBG 4917 produced 0.38 g of yeast per g of available substrate, grew at a rate of 3.18 g yeast produced per Litre per hour and had a protein content of about 60% was targeted by adding urea during the fed batch period.

### Growth on Palm Kernel Hydrolysate

Palm kernels are a by-product of the palm oil extraction industry and are readily hydrolysed using mannanase enzymes to produce a mixed sugar stream. MBG 4917 was grown on palm kernel hydrolysate supplemented with standard additions of trace elements, vitamins, and inorganic nutrients such as Mono-ammonium phosphate (MAP) and Urea.

The palm kernel hydrolysate contained the following organic compounds that MBG 4917 could readily consume.

TABLE 7

| Substrate | % w/v |
|---|---|
| Mannose | 8.44 |
| Glucose | 1.26 |
| Glycerol | 1.56 |

At the end of the fed-batch process the yeast was harvested and analysed. MBG 4917 had produced 0.45 g of yeast per g of available substrate, grew at a rate of 3.77 g yeast produced per Litre per hour and the protein content was 58.6%.

Unlike other strains of Saccharomyces cerevisiae, strain MBG 4917 is a yeast that can grow on a wider range of carbon sources and does so on minimal medium containing no additional nutrients.

In addition, this strain can accumulate very high levels of protein while growing on these carbon sources. As a result, it is suitable to produce yeast biomass having high protein content on waste streams such as biodiesel glycerol, glycerol rich syrups from corn ethanol plants and waste streams from the paper and pulp industries that are rich in both xylose and acetate.

### Example 7: Biomass Yield and Protein Levels in Fed Batch-Grown Yeast

Production of biomass under fed-batch conditions on corn ethanol stillage syrup, palm kernel hydrolysate and biodiesel glycerol (vegetable- and animal-derived)/cane molasses blend.

Strain MBG 4917 was grown on corn ethanol stillage syrup, palm kernel hydrolysate, and biodiesel glycerol (vegetable- and animal-derived feedstock)/cane molasses blend in separate fermenters.

Concentrated medium for fermentation experiments contained the carbon source detailed in Table 8 plus minimal mineral salts, trace metals, and vitamins required for normal growth of yeast were added (van Hoek et al., 2000 Biotechnol Bioeng. 68:517-23). Total utilizable carbon (TUC) refers to the total carbon compounds in the media on which strain MBG 4917 can grow.

TABLE 8

| Substrate | TUC (% w/v) |
|---|---|
| Corn ethanol stillage syrup 1 | 11.54 |
| Corn ethanol stillage syrup 2 | 14.80 |
| Corn ethanol stillage syrup 3 | 20.06 |
| Palm kernel hydrolysate 1 | 15.05 |
| Palm kernel hydrolysate 2 | 11.26 |
| Biodiesel glycerol (animal- | 30.49 |
| derived)/cane molasses blend | |
| Biodiesel glycerol (vegetable- | 31.68 |
| derived)/cane molasses blend | |

The total carbon compounds in the media on which stain MBG 4917 can grow were analysed using the high-performance liquid chromatography (HPLC).

TABLE 9
TUC composition of corn ethanol stillage syrup (% w/v)

| | | | | | Lactic | | |
|---|---|---|---|---|---|---|---|
| Substrate | Maltose | Fructose | Glucose | Xylitol | acid | Glycerol | Acetate |
| Corn ethanol | 1.70 | 0.49 | 0.81 | 0.29 | 1.47 | 6.57 | 0.21 |
| stillage syrup 1 | | | | | | | |
| Corn ethanol | 3.08 | 0 | 0.32 | 0 | 1.50 | 9.80 | 0.10 |
| stillage syrup 2 | | | | | | | |
| Corn ethanol | 0.67 | 0 | 0 | 0 | 1.52 | 17.74 | 0.13 |
| stillage syrup 3 | | | | | | | |

These corn ethanol stillage syrups were obtained from commercial dry-grind corn ethanol facilities and were representative of typical corn ethanol stillage.

TABLE 10
TUC composition of palm kernel hydrolysate (% w/v)

| Substrate | Mannose | Glucose | Glycerol | Acetate |
|---|---|---|---|---|
| Palm kernel hydrolysate 1 | 13.27 | 1.42 | 0.28 | 0.08 |
| Palm kernel hydrolysate 2 | 8.44 | 1.26 | 1.56 | 0 |

The palm kernel meal feedstock that was hydrolysed to produce the hydrolysate was sourced from Castlegate James, an Australian animal feed company. Mannanase enzyme was used to hydrolyse the palm kernel meal to produce a mixed sugar stream shown in the table above.

TABLE 11
TUC Composition of biodiesel glycerol/cane
molasses blend (% w/v)

| Substrate | Sucrose | Fructose | Glucose | Glycerol |
|---|---|---|---|---|
| Animal-derived biodiesel | 10.73 | 2.15 | 2.29 | 15.32 |
| glycerol/cane molasses | | | | |
| blend | | | | |
| Vegetable-derived | 10.51 | 2.85 | 3.21 | 15.11 |
| biodiesel glycerol/cane | | | | |
| molasses blend | | | | |

Nitrogen and phosphorus were added gradually during the fed batch period in the form of urea and monoammonium phosphate. Fermenters were internally controlled as follows: aeration 1-2 vvm (volume of air per unit volume of medium per minute), pH 4 to 6, temperature 30° to 32 °C for 20 to 30 hours depending on the growth rate of yeast in each substrate.

Analysis of the protein levels of the biomass showed high protein levels for strain MBG 4917 (Table 12).

TABLE 12

| | Yield, (g | Protein |
|---|---|---|
| Substrate | yeast / g TUC) | content (%) |
| Corn ethanol stillage syrup 1 | 0.30 | 60.0 |
| Corn ethanol stillage syrup 2 | 0.37 | 59.3 |
| Corn ethanol stillage syrup 3 | 0.38 | 58.2 |
| Palm kernel hydrolysate 1 | 0.33 | 65.3 |
| Palm kernel hydrolysate 2 | 0.45 | 58.6 |
| Biodiesel glycerol (animal-derived)/cane | 0.50 | 55.1 |
| molasses blend | | |
| Biodiesel glycerol (vegetable- | 0.42 | 53.2 |
| derived)/cane molasses blend | | |

The protein content of the yeast was measured using the Dumas combustion method.

The ability to grow yeast in scalable conditions defines the industrial application of strain MBG 4917. This example demonstrates the industrial utility of strain MBG 4917 and its growth on actual industrial waste stream substrates while still producing high levels of protein.

1-13. (canceled)

14. A Saccharomyces strain, wherein the strain:
(A) comprises the following characteristics:
(i) capable of growing on industrial waste with efficiency about the same as the Saccharomyces strain deposited under the Budapest Treaty and having NMI accession no. V23/009171 (strain MBG 4917) when grown under the same conditions; and
(ii) containing about the same amount of protein as strain MBG 4917 when grown under the same conditions, or
(B) is selected from:
(a) the Saccharomyces strain deposited under the Budapest Treaty and having NMI accession no. V23/009171 (strain MBG 4917); and
(b) a derivative of strain MBG 4917.

15. A Saccharomyces strain capable of growing on industrial waste and having a protein content of at least about 50% by weight.

16. The Saccharomyces strain according to claim 15, wherein the industrial waste is selected from the group consisting of corn ethanol stillage, palm kernel hydrolysate and biodiesel derived glycerol.

17. The Saccharomyces strain according to claim 15, wherein the strain is capable of growing on xylose, glycerol, acetate, lactic acid and/or ethanol, and having a protein content of at least 50% by weight.

18. The Saccharomyces strain according to claim 15, wherein a protein content is in the range of from about 50% to about 70% by weight.

19. The Saccharomyces strain according to claim 15, wherein the Saccharomyces strain is a non-genetically engineered Saccharomyces strain.

20. The Saccharomyces strain according to claim 14, wherein the strain is deposited under the Budapest Treaty and having NMI accession no. V23/009171 (strain MBG 4917).

21. A method of producing yeast having a protein content of at least 50% by weight, comprising incubating a Saccharomyces strain according to claim 14 with:
a substrate comprised of industrial waste, under conditions which promote growth of the Saccharomyces strain; or
a substrate comprising xylose, glycerol, acetate, lactic acid and/or ethanol, under conditions which promote growth of the Saccharomyces strain.

22. The method according to claim 21, wherein the industrial waste is selected from the group consisting of corn ethanol stillage, palm kernel hydrolysate and biodiesel derived glycerol.

23. An animal feed composition comprising a Saccharomyces strain according to claim 14.

24. The animal feed composition according to claim 23, wherein said Saccharomyces strain has a protein content of at least 50% by weight.

25. The animal feed composition according to claim 23, wherein said Saccharomyces strain has a protein content in the range of from about 50% to about 70% by weight.

26. The animal feed composition according to claim 23, wherein said Saccharomyces strain is a non-genetically engineered Saccharomyces strain.

27. The animal feed composition according to claim 23, wherein said Saccharomyces strain is deposited under the Budapest Treaty and has NMI accession no. V23/009171 (strain MBG 4917).

28. An animal feed composition comprising a Saccharomyces strain according to claim 15.

29. The animal feed composition according to claim 28, wherein said Saccharomyces strain has a protein content in the range of from about 50% to about 70% by weight.

30. The animal feed composition according to claim 28, wherein said Saccharomyces strain is a non-genetically engineered Saccharomyces strain.
